(19) United States
(12) Patent Application Publication
(10) Pub. No.: US 2007/0060756 A1
(43) Pub. Date: Mar. 15, 2007

(54) Process for the preparation of desloratadine

(75) Inventors: Veerappan Vijayabaskar, Chennai (IN); Siripragada Mahender Rao, Chennai (IN); Prabhakaran Vinod Kannan, Chennai (IN); Devasitham Sam Daniel Prabhu, Chennai (IN)

Correspondence Address: OLIFF & BERRIDGE, PLC, P.O. BOX 19928, ALEXANDRIA, VA, 22320, US

(73) Assignee: ORCHID CHEMICALS & PHARMACEUTICALS LTD., CHENNAI (IN)

(21) Appl. No.: 11/521,541

(22) Filed: Sep. 15, 2006

(30) Foreign Application Priority Data
Aug. 11, 2005 (IN) 1110/CHE/2005

(51) Int. Cl. C07D 401/02

(52) U.S. Cl. 546/93

(57) ABSTRACT

The present invention relates to an improved process for the preparation of compound of formula (I), 8-chloro-6,11-dihydro-11-(4-piperidinylidene)-5H-benzo[5,6]cyclohepta[1,2-b]pyridine (Desloratadine) or its pharmaceutically acceptable salts thereof, by reacting a compound of formula (II), 4-(8-Chloro-5,6-dihydro-11H-benzo[5,6]cyclohepta[1,2-b]pyridin-11-ylidene)-1 piperidinecarboxylic acid ethyl ester (Loratadine) with sodium hydroxide in a solvent mixture of toluene and polyethylene glycol (PEG 400) at reflux temperature.

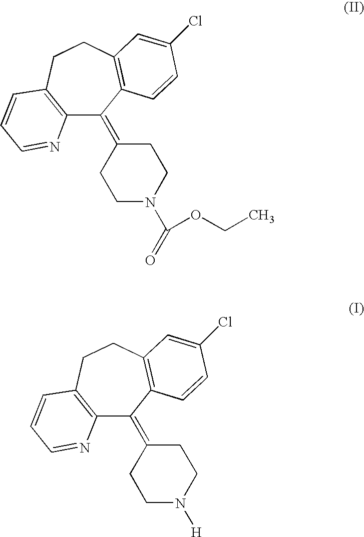

### FIELD OF THE INVENTION

The present invention relates to an improved process for the preparation of compound of formula (I), 8-chloro-6,11-dihydro-1-(4-piperidinylidene)-5H-benzo[5,6]cyclohepta[1,2-b]pyridine (Desloratadine) or its pharmaceutically acceptable salts thereof, by reacting a compound of formula (II), 4-(8-Chloro-5,6-dihydro-11H-benzo[5,6]cyclohepta[1,2-b]pyridin-11-ylidene)-1 piperidinecarboxylic acid ethyl ester (Loratadine) with sodium hydroxide in a solvent mixture of toluene and polyethylene glycol (PEG 400) at reflux temperature.

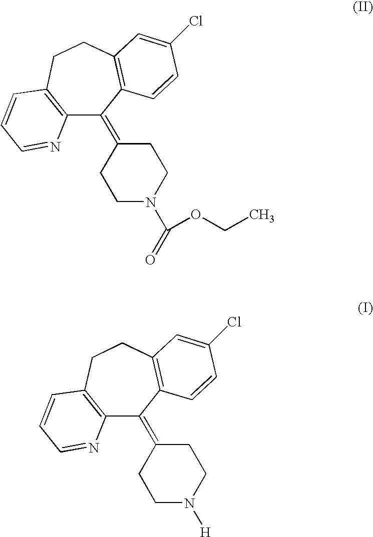

### BACKGROUND OF THE INVENTION

Desloratadine, known as 8-chloro-6,11-dihydro-11-(4-piperidylidene)-5H-benzo[5,6]cyclohepta[1,2-b]pyridine, has the following structure:

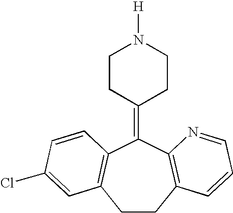

Desloratadine is currently marketed as Clarinex in the United States. Clarinex is prescribed as an antihistamine for prevention or treatment of allergenic reactions, which may result in symptoms such as sneezing, itchy eyes and hives.

U.S. Pat. No. 4,659,716 (hereinafter '716) discloses descarbonylethoxyloratadine (also known as Desloratadine), which possesses antihistaminic properties with substantially no sedative properties. The '716 patent describes a process for the preparation of Desloratadine by dissolving loratadine in water and basifying with dilute solution of potassium carbonate to obtain a pink coloured oil. The organic material is extracted with chloroform, washed with water and triturated with hexane. Desloratadine is obtained by recrystallisation of the extracted organic material with large volume of hexane after charcolisation.

U.S. Pat. No. 6,506,767 (hereinafter '767) discloses two polymorphic forms of desloratadine, labeled Forms I and II. The XRPD peaks and the FTIR spectrum for the forms are also disclosed in the '767 patent. According to this patent '767 patent, discloses certain alcoholic solvents, e.g., hexanol and methanol produce 100% polymorph form 1, but others, e. g., 3-methyl-1-butanol and cyclohexanol produce significant amounts of form 2. Chlorinated solvents, e. g., dichloromethane produce form 1 substantially free of form 2. Ether solvents such as dioxane produced form 1 substantially free of form 2 but other alkane ethers, e.g., di-isopropyl ether produced form 1 with significant amounts of form 2 and di-n-butyl ether favored formation of form 2. Ketones such as methyl isobutyl ketone produced crystalline polymorph form 1 essentially free of form 2 but methyl butyl ketone produced 8:1 ratio of form 1 to form 2. Use of methyl isobutyl ketone is preferred to produce crystalline polymorph form 1 essentially free of form 2. Only ethyl acetate and di-n-butyl ether were found to produce crystalline polymorph form 2 substantially free of form 1. Use of di-n-butyl ether is preferred for producing crystalline form 2 substantially free of form 1. According to this patent the polymorph form obtained from U.S. Pat. No. 4,659,716 is a mixture of form I and form II.

Teva Patent WO2004/080461 claims a pharmaceutical composition of desloratadine comprising of a mixture of crystalline Desloratadine of form I and II in a weight to weight ratio of about 25% to about 75% of either form to the other and a pharmaceutically acceptable excipient.

None of the prior art references discloses or claims the use of solvent mixture toluene and polyethylene glycol (PEG 400) for hydrolysis of Loratadine.

We focused our research to develop an improved and efficient process for the preparation of the compound of formula (I) in good yield and high purity. The disclosed process has advantages over the processes described in the above-mentioned prior art documents.

### OBJECTIVES OF THE INVENTION

The main objective of the present invention is to provide an improved process for the preparation of compound of formula (I) and its pharmaceutically acceptable salts thereof, using sodium hydroxide for hydrolysis of compound of formula (II) in the presence of solvent mixture toluene and polyethylene glycol (PEG 400).

Another objective of the present invention is to provide a process for the preparation of compound of formula (I) in polymorphic mixture of Form I and II and its pharmaceutically acceptable salts in good yield and high purity.

### SUMMARY OF THE INVENTION

Accordingly, the present invention provides an improved process for the preparation of Desloratadine of formula (I) and its pharmaceutically acceptable salts, comprising reacting 4-(8-Chloro-5,6-dihydro-11H-benzo[5,6]cyclohepta[1,2-b]pyridin-11-ylidene)-1-piperidine carboxylic acid ethyl ester of formula (II) (Loratadine), with alkali hydroxide in a solvent mixture of aromatic hydrocarbon and polyethylene glycol

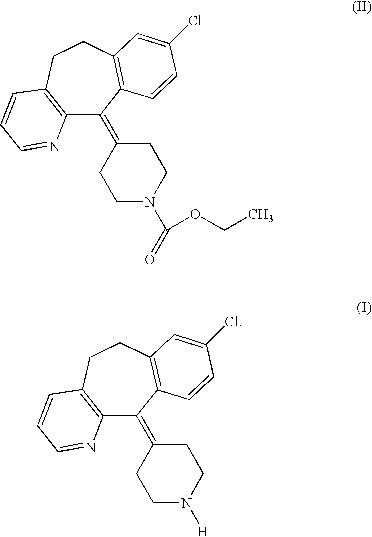

In this regard, a desirable process for preparing Desloratadine of formula (I) and its pharmaceutically acceptable salts comprises

i.) reacting 4-(8-Chloro-5,6-dihydro-11H-benzo[5,6]cyclohepta[1,2-b]pyridin-11-ylidene)-1-piperidinecarboxylic acid ethyl ester (Loratadine) with sodium hydroxide in a solvent mixture of toluene and polyethylene glycol (PEG 400) at reflux temperature;

ii) adding a mixture of water and toluene to the reaction mixture, whereby two phases are obtained;

iii) separating the organic phase;

iv) concentrating the separated organic phase;

v) dissolving the obtained concentrate in a toluene: methyl isobutyl ketone (MIBK) mixture followed by the addition of isopropyl ether (IPE) at 60° C.;

vi) cooling the slurry mass to 5° C. to 10° C.;

vii) isolating Desloratadine by filtration.
The process is shown in the scheme given below

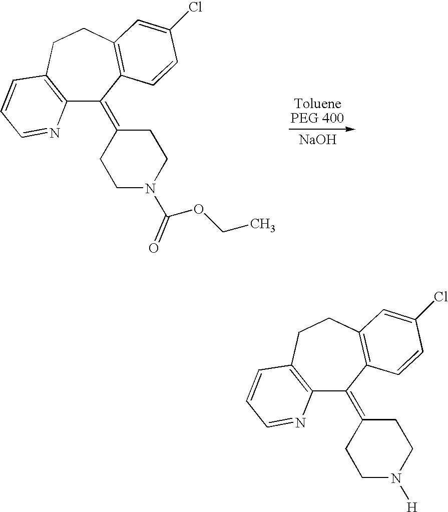

### DETAILED DESCRIPTION OF THE INVENTION

In an embodiment of the present invention, the reaction step is performed in a solvent. The solvent is a mixture of an aromatic hydrocarbon and polyethylene glycol, and may be selected from the group consisting of toluene, xylene and polyethylene glycol (PEG 400) or mixtures thereof. The most preferred solvent mixture for this reaction is toluene with polyethylene alcohol (PEG 400).

In another embodiment of the present invention, the reacting step is preferably performed at a temperature of about 45° C. to about 160° C. Most preferably; the reaction step is performed at a temperature of about 100° C. to about 160° C.

In yet another embodiment of the present invention the starting material of this invention is prepared according to the literature available in the prior art.

In still another embodiment of the present invention the compound of formula (I) obtained is in the mixture of polymorph I and II, wherein one of the polymorph is in the range of less than 25% over other and vice versa.

In a further embodiment, a process for purifying Desloratadine comprises dissolving crude Desloratadine in a mixture of toluene/methyl isobutyl ketone (MIBK); adding anti-solvent to the mixture from a) and isolating pure Desloratadine. A desirable anti-solvent is isopropyl ether (IPE).

The present invention is exemplified by the following example, which is provided for illustration only and should not be construed to limit the scope of the invention.

### EXAMPLE 1

### Step-i-Preparation of 8-chloro-6,11-dihydro-11-(4-piperidinylidene)-5H-benzo[5,6]cyclohepta[1,2-b]pyridine (Desloratadine)

To a solution of loratadine (50 gms) in toluene (200 ml), polyethylene glycol 400 (100 ml) was added and stirred. Sodium hydroxide (50 gms) was added to the above reaction mixture and refluxed for 2 hours. The course of the reaction was monitored by HPLC and after completion of the reaction, water (750 ml) and toluene (100 ml) was added and stirred well. The organic layer was separated out; the aqueous layer was extracted with toluene (2×100 ml). The combined organic layer was washed with water (2×250 ml) followed by brine (1×250 ml) and evaporated the solvent under vacuum.

Recrystallization

The obtained solid was dissolved in the mixture of toluene: methylisobutyl ketone (1:1 v/v) at 85° C., followed by the addition of IPE at 60° C. and cooled to 5° C. to 10° C. The obtained solid was filtered off. This recrystallization may be repeated to obtain the desired quality and purity of Desloratadine.

Yield: 19.0 gms (˜48.0%).

We claim:

1. A process for preparing Desloratadine of formula (I), comprising reacting 4-(8-Chloro-5,6-dihydro-11H-benzo[5,6]cyclohepta[1,2-b]pyridin-11-ylidene)-1-piperidine carboxylic acid ethyl ester of formula (II) (Loratadine), with alkali hydroxide in a solvent mixture of aromatic hydrocarbon and polyethylene glycol

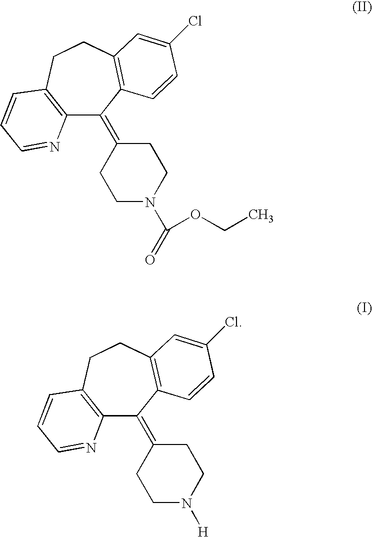

2. A process according to claim 1, wherein the alkali hydroxide is sodium hydroxide.

3. A process according to claim 1, wherein the solvent mixture is toluene and polyethylene glycol 400.

4. A process for purifying Desloratadine comprising
a) dissolving crude Desloratadine in a mixture of toluene/methyl isobutyl ketone (MIBK);
b) adding anti-solvent to the mixture from a) and
c) isolating pure Desloratadine of formula (I)

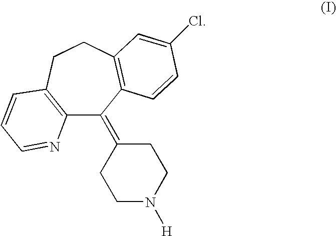

5. A process according to claim 4, wherein the anti-solvent is isopropyl ether (IPE).
